(19) United States
(12) Patent Application Publication
(10) Pub. No.: US 2005/0187568 A1
(43) Pub. Date: Aug. 25, 2005

(54) Devices and methods for closing a patent foramen ovale with a coil-shaped closure device

(76) Inventors: Alan R. Klenk, San Jose, CA (US); Hai Q. Le, San Jose, CA (US); Thuzar K. Han, Fremont, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/783,783

(22) Filed: Feb. 20, 2004

(51) Int. Cl. A61B 17/08

(52) U.S. Cl. 606/153

(57) ABSTRACT

A patent foramen ovale closure device and method are provided. The device is deployed at the foramen ovale to secure the septum primum and septum secundum together, thus sealing the patent foramen ovale. In one embodiment, the closure device is a coil that is delivered through the septum secundum and septum primum in an axially elongated state using a tissue piercing structure. After being delivered, the coil is released, causing the coil to axially shorten to pinch the septum primum and septum secundum together.

### BACKGROUND OF THE INVENTION

1. Field of the Invention

The present invention relates in certain embodiments to methods and devices for closing a body lumen or cavity and, in particular, for closing a patent foramen ovale.

2. Description of the Related Art

Embolic stroke is the nation's third leading killer for adults, and is a major cause of disability. There are over 700,000 strokes per year in the United States alone. Of these, roughly 100,000 are hemorrhagic, and 600,000 are ischemic (either due to vessel narrowing or to embolism). About 50,000 of the ischemic strokes are believed to be caused by a patent foramen ovale. However, the risk of recurrent stroke is higher in patients whose strokes are caused by a patent foramen ovale.

Pharmacological therapies for stroke prevention such as oral or systemic administration of warfarin or the like have been inadequate due to serious side effects of the medications and lack of patient compliance in taking the medication.

In general, the heart is divided into four chambers, the two upper being the left and right atria and the two lower being the left and right ventricles. The atria are separated from each other by a muscular wall, the interatrial septum, and the ventricles by the interventricular septum.

Either congenitally or by acquisition, abnormal openings, holes or shunts can occur between the chambers of the heart or the great vessels (interatrial and interventricular septal defects or patent ductus arteriosus and aortico-pulmonary window respectively), causing shunting of blood through the opening. During fetal life, most of the circulating blood is shunted away from the lungs to the peripheral tissues through specialized vessels and foramens that are open (“patent”). In most people these specialized structures quickly close after birth, but sometimes they fail to close. A patent foramen ovale is a condition wherein an abnormal opening is present in the septal wall between the two atria of the heart. An atrial septal defect is a condition wherein a hole is present in the septal wall between the two atria of the heart.

In contrast to other septal defects which tend to have a generally longitudinal axis, a patent foramen ovale tends to behave like a flap valve. Accordingly, the axis of the patent foramen ovale tends to be at an angle, and almost parallel to the septal wall. The patent foramen ovale is a virtual tunnel, long and wide, but not very tall. It is normally closed because the roof and floor of the tunnel are in contact, but it can open when the pressure in the right side of the heart becomes elevated relative to the pressure in the left side of the heart, such as while coughing

Studies have shown that adults with strokes of unknown origin (cryptogenic strokes) have about twice the normal rate of patent foramen ovales than the normal population. Although there is a correlation between strokes and patent foramen ovales, it is currently unknown why this correlation exists. Many people theorize that blood clots and plaque that have formed in the peripheral venous circulation (in the legs for example) break off and travel to the heart. Normally, the clots and plaque get delivered to the lungs where it is trapped and usually cause no harm to the patient. Patients with a patent foramen ovale, however, have a potential opening that the clots or plaque can pass through the venous circulation and into the arterial circulation and then into the brain or other tissues to cause a thromboembolic event like a stroke. The clots may pass to the arterial side when there is an increase in the pressure in the right atrium. Then the clots travel through the left side of the heart, to the aorta, and then to the brain via the carotid arteries where they cause a stroke and the associated neurological deficits.

Previously, patent foramen ovale have required relatively extensive surgical techniques for correction. To date the most common method of closing intracardiac shunts, such as a patent foramen ovale, entails the relatively drastic technique of open-heart surgery, requiring opening the chest or sternum and diverting the blood from the heart with the use of a cardiopulmonary bypass. The heart is then opened, the defect is sewn shut by direct suturing with or without a patch of synthetic material (usually of Dacron, Teflon, silk, nylon or pericardium), and then the heart is closed. The patient is then taken off the cardiopulmonary bypass machine, and then the chest is closed.

In place of direct suturing, closure of a patent foramen ovale by means of a mechanical prosthesis has also been disclosed. A number of devices designed for closure of interauricular septal defects have been used to correct patent foramen ovale.

Although these devices have been known to effectively close other septal defects, there are few occlusion devices which have been developed specifically for closing patent foramen ovale. Although these devices have been effective in some cases, there is still much room for improvement.

Notwithstanding the foregoing, there remains a need for a transluminal method and improved apparatus for correcting patent foramen ovale.

### SUMMARY OF THE INVENTION

Embodiments of the present invention provide a minimally invasive closure device for closing a patent foramen ovale. Improved delivery and positioning systems are also provided.

In one embodiment, a method of closing a patent foramen ovale having a septum primum and a septum secundum is provided. An elongate body having a proximal end and a distal end is delivered to the patent foramen ovale. The elongate body has a tissue piercing structure at its distal end and a coil releasably engaged with the elongate body. The tissue piercing structure and the coil are advanced through the septa of the patent foramen ovale. The coil is released from the elongate body and the tissue piercing structure is withdrawn from the septa of the patent foramen ovale, the coil when released contracting to pinch the septum primum and the septum secundum together.

The elongate body may include an opening near its distal end, and the coil may have a distal end that releasably engages the opening in the elongate body near its distal end. A loading portion may be provided to releasably engage a proximal end of the coil, the coil being advanced through the septa of the patent foramen ovale while the coil is engaged with both the loading portion and the opening near the distal end of the elongate body to axially elongate and radially reduce the coil. In one embodiment, a loading collar is delivered with the elongate body to the patent foramen ovale, the loading collar releasably engaging a proximal end of the coil. The elongate body may be rotatable relative to the loading collar, and/or may be axially slideable relative to the loading collar.

In another embodiment, a method of closing a patent foramen ovale having a septum primum and septum secundum comprises advancing a plurality of coils at least partially through the septa of the patent foramen ovale to secure the septum primum and septum secundum together. The plurality of coils may be advanced sequentially through a single catheter. Each of the coils may be provided over a single elongate body and be advanced through the septa of the patent foramen ovale using a tissue piercing structure on the distal end of the elongate body. In one embodiment, at least three coils are advanced through the septa of the patent forman ovale.

In another embodiment, an assembly for delivering a coil through tissue in a patient is provided. The assembly comprises a loading portion adapted to releasably engage a proximal end of the coil. A tissue piercing structure is also provided adapted to releasably engage a distal end of the coil. The loading portion holds the coil relative to the tissue piercing structure to axially elongate and radially reduce the coil. In one embodiment, the loading portion is integral with the tissue piecing structure. The loading portion may have a rectangular shape. The loading portion may also comprise a slot adapted to receive the proximal end of the coil. In one embodiment, the loading portion comprises a loading collar, and the tissue piecing structure can be rotated relative to the loading collar. Further, the tissue piercing structure can be moved axially relative to the loading collar to axially elongate the coil.

### BRIEF DESCRIPTION OF THE DRAWINGS

FIG. 1 is an anterior illustration of a heart, with the proximal parts of the great vessels.

FIG. 2 is a perspective view of a rotatable closure device in accordance with one embodiment of the present invention.

FIG. 2A is a perspective view of an alternative embodiment of a rotatable closure device in accordance with one embodiment of the present invention.

FIG. 3A is a perspective view of a closure device in accordance with one embodiment of the present invention.

FIG. 3B is an end view of the closure device of FIG. 3A.

FIG. 3C is a perspective view of an alternative embodiment of the closure device of FIG. 3A.

FIG. 4 is a schematic view of a closure device delivery system.

FIG. 5 is a detailed perspective view of the distal end of the closure device delivery system in accordance with one embodiment of the present invention.

FIG. 6 is a perspective view of a closure device delivery system in accordance with one embodiment of the present invention.

FIG. 7 is a perspective view of a loading collar used in the delivery system of FIG. 6.

FIGS. 8-13 are schematic views illustrating a closure procedure in accordance with one embodiment of the present invention.

FIG. 14 is a perspective view of a closure device delivery system adapted to deliver a plurality of closure devices.

FIGS. 15A and 15B are schematic views of a closure device delivered through a patent foramen ovale.

FIG. 16. is a schematic view illustrating a plurality of closure devices delivered through the septa of a patent foramen ovale.

FIGS. 17A and 17B are partial cross-sectional views of a closure device delivery system in accordance with another embodiment of the present invention.

### DETAILED DESCRIPTION OF THE PREFERRED EMBODIMENTS

For simplicity, the embodiments of the present invention will be described primarily in the context of a patent foramen ovale closure procedure. However, the device and methods herein are readily applicable to a wider variety of closure or attachment procedures, and all such applications are contemplated by the present inventors. For example, additional cardiac procedures such as atrial septal defect closure, ventricular septal defect closure, and atrial appendage closure are contemplated. Vascular procedures such as patent ductus arteriosis closure, isolation or repair of aneurysms, anastamosis of vessel to vessel or vessel to prosthetic tubular graft joints may also be accomplished using the devices as described herein. Attachment of implantable prostheses, such as attachment of the annulus of a prosthetic tissue, mechanical heart valve or an annuloplasty ring may be accomplished. A variety of other tissue openings, lumens, hollow organs and surgically created passageways may be closed in accordance with the preferred embodiments. Closures and repairs described herein may be accomplished using catheter based interventional methods or minimally invasive surgical methods. Adaptation of the devices and methods disclosed herein to accomplish procedures such as the foregoing will be apparent to those of skill in the art in view of the disclosure herein.

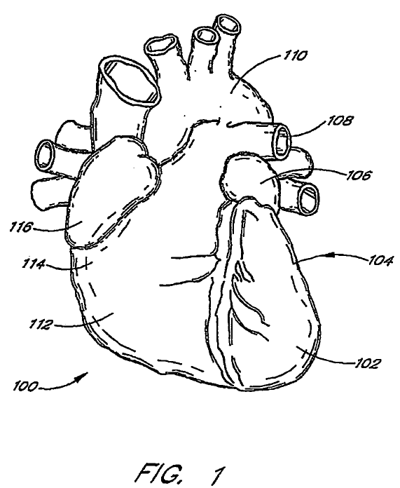

Referring to FIG. 1, a heart 100 is illustrated to show certain portions including the left ventricle 102, the left atrium 104, the left atrial appendage 106, the pulmonary artery 108, the aorta 110, the right ventricle 112, the right atrium 114, and the right atrial appendage 116. As is understood in the art, the left atrium 104 is located above the left ventricle 102 and the two are separated by the mitral valve (not illustrated).

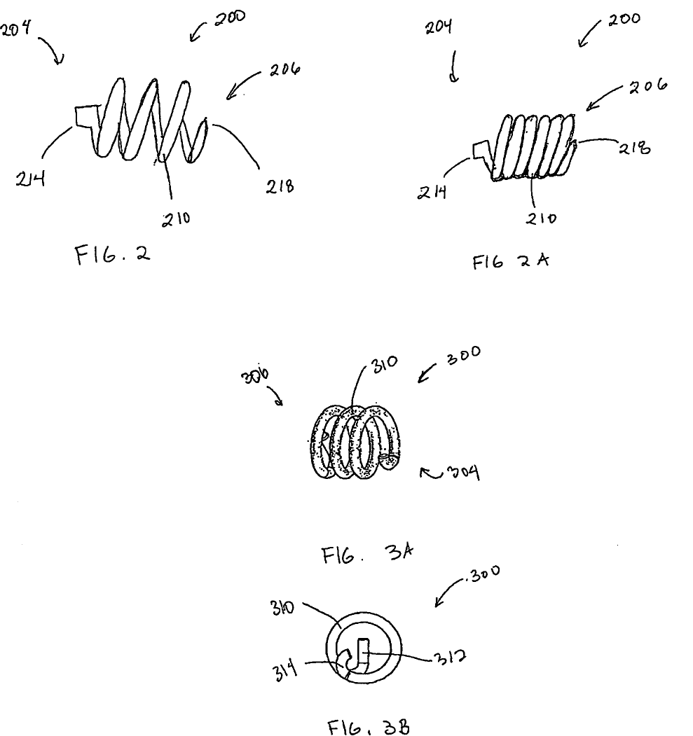

With reference to FIG. 2, a rotatable patent foramen ovale closure device is shown. In these embodiments, the patent foramen ovale is simply held together by positioning a device 200 to hold the septum primum and septum secundum together. The device 200 comprises a proximal end 204 and a distal end 206. The device has a spring-like configuration and comprises a coiled wire 210. The device 200 may include a detachment element 214 at its proximal end which may comprise a loop, internal threading, external threading, or other structures adapted to releasably engage the device 200 to a delivery device. The distal end 206 may include a sharpened point 218 for puncturing the tissue. The device may also be provided with a sleeve. FIG. 2A shows an embodiment of the device 200 wherein the closure device has a pitch less than the pitch shown in the closure device of FIG. 2.

In one embodiment, rotatable closure device 200 may have a left-handed threading. In another embodiment, rotatable closure device 200 may have a right-handed threading. In some embodiments, the coil 210 may have a variable pitch. In some embodiments, the diameter of the rotatable closure device may vary along the length of the device. In one embodiment, the coil has a diameter of about ⅛ to ½ inch.

To deliver the device 200, in one embodiment the proximal end 204 is positioned in the right atrium, while the distal end 206 is positioned in the left atrium, by rotating the device 200 through the septum secundum and septum primum using a delivery device releasably attached to the device 200 at detachment element 214. It is also envisioned that the proximal end 204 may be positioned in the left atrium, while the distal end 206 may be positioned in the right atrium, by rotating the device 200 through the septum primum and septum secundum. After delivery, the delivery device can be detached from detachment element 214. The device may be delivered such that after passing through the septum primum and septum secundum, the coil axially shortens due to its natural pitch, thereby pinching the septum primum and septum secundum together. In another embodiment, the coil may tend to axially shorten towards an unstressed shape due to the elasticity of the coil material.

Preferably, the device 200 is formed of a metal such as stainless steel, Nitinol, Elgiloy, or others which can be determined through routine experimentation by those of skill in the art. The wire may also be biodegradable. Wires having a circular or rectangular cross-section may be utilized depending upon the manufacturing technique. Wires may be stranded or cabled. In one embodiment, a circular cross section wire is cut such as by known laser cutting techniques from tube stock. In another embodiment the wire coil is substantially formed on a coil winding machine. The closure device is preferably an integral structure, such as a single ribbon or wire, or element cut from a tube stock. In some embodiments the wire coil is made of Nitinol and heat set to a pre-determined shape which the coil tends to assume following coil deployment or implantation.

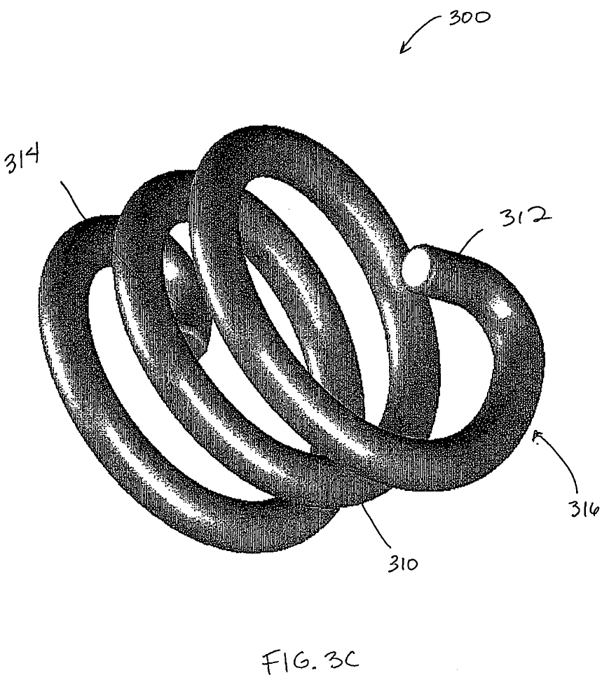

FIGS. 3A and 3B show another embodiment of a closure device in accordance with one embodiment of the present invention. The device 300 comprises a proximal end 304 and a distal end 306. The device may be a coil or have a helical spring-like configuration as described above, and as illustrated comprises a coiled wire 310. It will be appreciated that the term “coil” is a broad term and is used herein in its ordinary sense and includes, without limitation, coils, helical wires or ribbons, springs, or any other similarly shaped structure. The coiled wire is bent inward at its proximal end 304 and distal end 306 as shown in FIG. 3B, to form a proximal tang 314 and distal tang 312, respectively. FIG. 3C shows an embodiment of the device 300 wherein a bend 316 is provided in the coiled wire 310 near distal tang 312.

Examples of other coil shapes can be found in U.S. Pat. Nos. 5,810,882 and 5,582,616, the entireties of which are hereby incorporated by reference.

In one embodiment, the closure device 300 may be made from a medical plastic or a metal, such as stainless steel, Nitinol, Elgiloy, polyester, PEEK or others which can be determined through routine experimentation by those of skill in the art. In another embodiment, the closure member 300 may be made of a dissolvable suture material. The closure device 300 may also be biodegradable. It is also envisioned that other metallic or non-metallic biocompatible materials may be used to form closure device 300. In one preferred embodiment, the closure device is a superelastic coil, which can be radially compressed and axially expanded from its natural, relaxed state to its stressed state.

In some embodiments, the closure device may be coated with a thin layer of a tissue ingrowth material, such as collagen, polyester, ceramic, and the like. The coating may be porous, such as a porous hydroxyapatite material. A Dacron, polyester, or other tissue growth prompting or accepting material may be used with the closure device. In one embodiment, at least a portion of the closure device may be coated with a fabric comprising the tissue ingrowth material. In one embodiment, the closure device may comprise a coating over at least a portion of the device. The closure device may be manufactured in any of a variety of ways, such as machining, molding, and the like.

The coiled wire 310 may have a circular, rectangular, or other shaped cross-section, depending upon the manufacturing technique. In one embodiment, a circular cross section is molded from a biocompatible polymer, such as polyethylene terephthalate (PET). In some embodiments, the closure device 300 may have any pitch or a variable pitch. In some embodiments, the diameter of the closure device may vary longitudinally.

For use in a patent foramen ovale, in one embodiment, the device 300 has an outer diameter D having any value or range of values from about 0.005 in to about 0.375 in, and, in one more preferred embodiment, about 0.11 in. The overall length L of the closure device 300 from the distal end 306 to the proximal end 304 in one embodiment is any value or range of values from about 0.040 to 0.120 in. In some embodiments, the wire has a diameter of any value or range of values between about 0.005-0.02 in, and, in one some preferred embodiments, any value or range of values between about 0.008-0.014 in, and in one more preferred embodiment, about 0.010 in.

In some embodiments, radiopaque markers may be provided on the closure device 300 to aid in placement at the treatment site. In some embodiments, the radiopaque markers are crimped on to the closure device. In one embodiment, the radiopaque markers are tubular bands crimped on to the closure device. In some embodiments, the radiopaque markers are coatings applied to the device. In some embodiments, the radiopaque markers may be platinum or iridium, and the like. In some embodiments the radiopaque marker is a wire core, wire coating, or wire strand of radiopaque material.

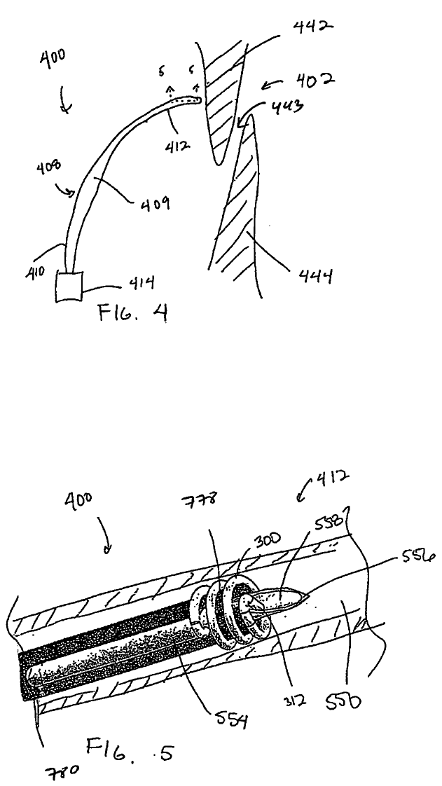

Referring to FIG. 4, a delivery device 400 may be used to deliver the closure device to the patent foramen ovale 402. The patent foramen ovale 402 generally includes a septum primum 444 and a septum secundum 442 and a tunnel 443 extending therethrough. The delivery device 400 comprises a catheter 408 having an elongate flexible tubular body 409 extending between a proximal end 410 and a distal end 412. The catheter is shown in a highly schematic form, for the purpose of illustrating the functional aspects thereof. The catheter body will have a sufficient length and diameter to permit percutaneous entry into the vascular system, and transluminal advancement through the vascular system to the desired deployment site. For example, in an embodiment intended for access at the femoral artery and deployment within the right atrium, the catheter 408 will have a length within the range of from about 50 cm to about 150 cm, and a diameter of generally no more than about 15 French. Further dimensions and physical characteristics of catheters for navigation to particular sites within the body are well understood in the art and will not be further described herein.

The flexible body can be manufactured in accordance with any of a variety of known techniques. In one embodiment, the flexible body 409 is extruded from any of a variety of materials such as HDPE, PEBAX, nylon, and PEEK. Alternatively, at least a portion of or all of the length of the tubular body may comprise a spring coil, solid walled hypodermic needle or other metal tubing, or a braided reinforced wall, as are known in the art. The spring coil, tubing, braided reinforcement, or other structures may be encapsulated with thermoset polymers such as polyimide or with thermoplastic polymers such as PEBAX, and the like.

The tubular body 409 may be provided with a handle 414 generally on the proximal end 410 of the catheter 408. The handle 414 may be provided with a plurality of access ports. The handle 414 may be provided with an access port which may be used as a guide wire port in an over the wire embodiment, and a deployment wire port. Additional access ports, such as a contrast media introduction port, or others may be provided as needed, depending upon the functional requirements of the catheter. The catheter 408 may be constructed to contain the same number of ports as the handle 414. The handle 414 permits manipulation of the various aspects of the occlusion device delivery system 400, as will be discussed below. Handle 414 may be manufactured in any of a variety of ways, typically by injection molding, machining or otherwise forming a handpiece for single-hand operation, using materials and construction techniques well known in the medical device arts.

With reference to FIG. 5, the catheter 408 may include a passageway 550 for delivery of the closure device 300 to the patent foramen ovale. An elongate body may be delivered through the passageway 550, and may include a tissue piercing structure 554, such as a needle. The tissue piercing structure 554 may have a generally tubular body and is moveable axially and rotationally relative to the catheter 408. In one embodiment, the tissue piercing structure may be spring-loaded to advance upon actuation to a location distal of the distal end 412 of the catheter. The tissue piercing structure 554 may have a pointed end 556 for accessing the patent foramen ovale, as will be described below. At the distal end of the tissue piercing structure 554, an opening 558 may be provided in which the distal end 306 of the closure device 300 engages. More preferably, the distal tang 312 of the closure device 300 engages with opening 558. As illustrated in FIG. 5, a loading portion for the proximal tang 314 is provided in the form of a loading collar 778 over the tissue piercing structure 554. Loading collar 778 includes an opening 780, more preferably a longitudinal slot, which releasably engages the proximal end 304 of the closure device, more preferably engaging proximal tang 314.

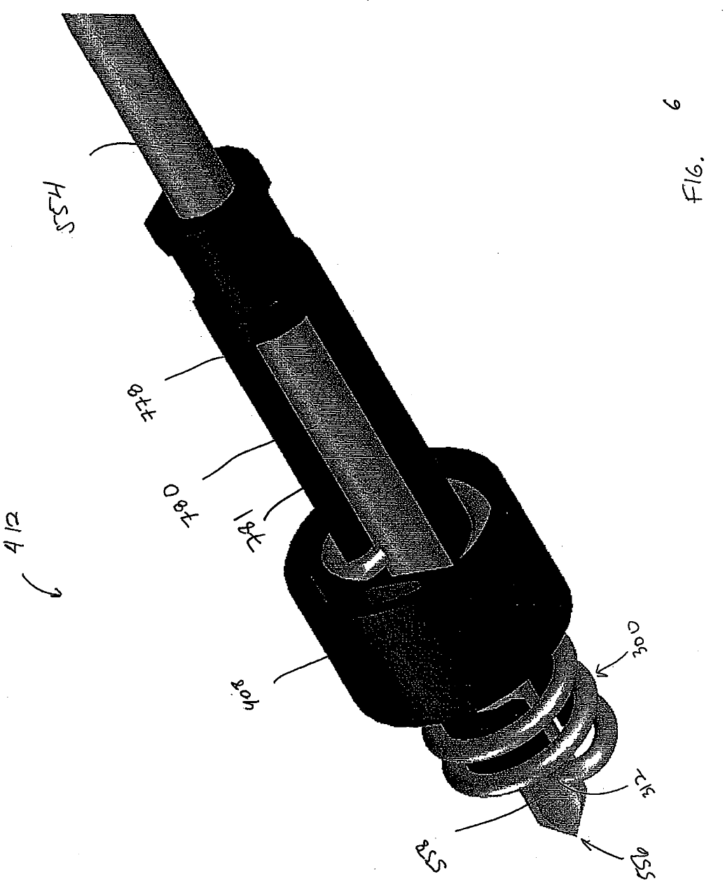

FIGS. 6 and 7 illustrate in perspective view one embodiment of a tissue piercing structure 554 with a loading collar 778 provided thereover. As shown in FIG. 6, the tissue piercing structure 554 may have a pointed end 556 for accessing the patent foramen ovale, and has an opening 558 for receiving distal tang 312 of the closure device 300. The tissue piercing structure 554 may be axially slideable and rotatable relative to loading collar 778, which releasably engages a proximal tang 314 as described above. The catheter 408 is illustrated in a partially cut-away view, having a plurality of lumens extending therthrough.

Loading collar 778 is preferably an elongate tubular body which may extend to the proximal end of catheter 408, or may be operated from the handle 414 using a suitable actuator extending through the catheter 408. For example, the loading collar may include a proximal flexible section that extends to the proximal end of the catheter 408. In one embodiment, the loading collar is rotatable about ¼ of a turn relative to the tissue piercing structure.

An elongate opening or slot 780 extends longitudinally along the loading collar, and as shown in FIG. 7, includes a projection 786 toward a distal end thereof, near the pointed end of the tissue piercing structure. The opening 780 and projection 786 form a track 788 which is used to guide and release the proximal tang 314 (as well as the distal tang 312 in the multiple coil embodiment, described below) of the closure device 300 from the loading collar 778 for deployment of the closure device, as described below. The track 788 is shown having a generally vertical portion 790 which is generally parallel to the projection 786 and a generally horizontal portion 792 which is generally perpendicular to the vertical portion 790 and parallel to the longitudinal axis of the opening 780. The horizontal portion 792 of the track 788 extends to the distal end of the loading collar 778.

In some embodiments, as shown in FIG. 6, the closure device 300 is loaded by engaging distal tang 312 with the opening 558 on tissue piercing structure 554, and wrapping the turns of the coils of closure device 300 over the loading collar 778, which extends proximally from the opening 558. The proximal tang 314 engages opening 780 in loading collar 778 proximal to projection 786. Rotating the tissue piercing structure 554 relative to the loading collar 778, for example clockwise (when viewed from the proximal end of the device), and axially advancing the tissue piercing structure 554 relative to the loading collar 778, causes the proximal tang to rotate and move distal to the projection 786. With continued clockwise rotation of the tissue piercing structure 554 relative to the loading collar 778, the long wall 781 of the opening 780 engages the tang 314 to radially compress the device 300 around the cylindrical body of the tissue piercing structure 554. The device loaded in this configuration may be advanced through tissue, as described below.

To release the closure device 300 from the tissue piercing structure 554 and loading collar 778, the tissue piercing structure 554 may be rotated, for example counter-clockwise, thereby allowing the coil to achieve its natural diameter in the tissue. The tissue piercing structure may then be axially retracted relative to the loading collar 778, pulling the tissue piercing structure from the tissue, as described below, and proximally through the device 300. Finally, the loading collar 778 may be rotated, for example clockwise, until the proximal tang 314 snaps out of the track 788, sliding along the vertical portion 790 and out horizontal portion 792.

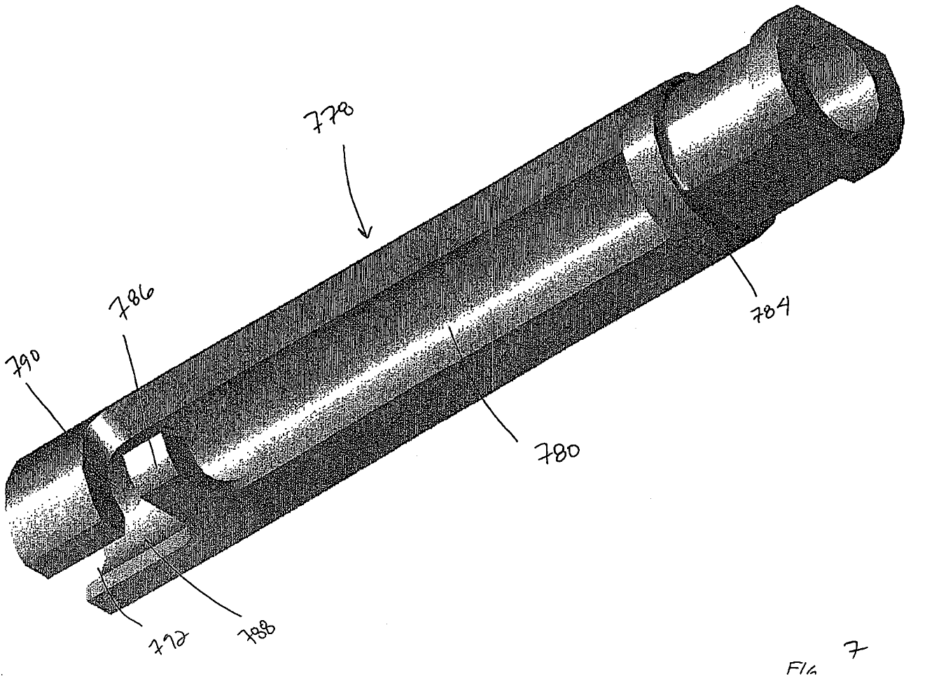

A method of delivering the closure device 300 to a treatment site, such as a patent foramen ovale, is shown in FIGS. 8-13. In use, the delivery device 400 is percutaneously introduced into the vascular system and transluminally advanced into the heart and, subsequently, to the patent foramen ovale using techniques which are known in the art.

The patent foramen ovale may be accessed via catheter through a variety of pathways. In one embodiment, the patent foramen ovale may be accessed from the venous circuit. The catheter may be introduced into the venous system, advanced into the inferior vena cava or superior vena cava and guided into the right atrium. The catheter may then be directed to the patent foramen ovale. Alternatively, once in the right atrium, the catheter may be advanced through the tricuspid valve and into the right ventricle and directed to a ventricular septal defect and the closure device deployed.

Alternatively, the patent foramen ovale may be accessed from the arterial circuit. The catheter is introduced into the arterial vascular system and guided up the descending thoracic and/or abdominal aorta. The catheter may then be advanced into the left ventricle through the aortic outflow tract. Once in the left ventricle, the catheter may be directed up through the mitral valve and into the left atrium. When the catheter is in the left atrium, it may be directed into the patent foramen ovale and the closure device deployed.

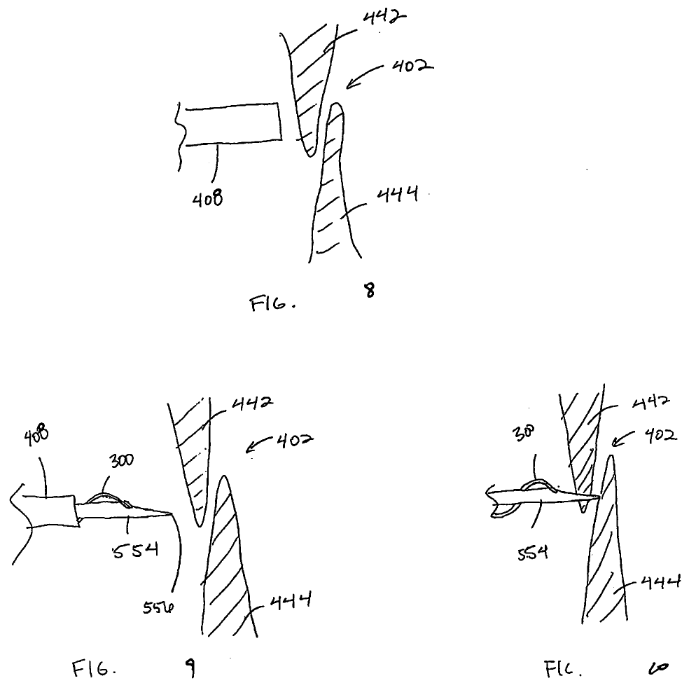

As shown in FIG. 8, a catheter 408 approaches a patent foramen ovale from the right atrium. Initially, the closure device 300 is configured inside the catheter 408. As shown in FIG. 9, the tissue piercing structure 554, which releasably engages distal tang 312 of closure device 300, is advanced out of the catheter 408. Loading collar 778 (not shown in FIG. 9) may also be advanced out of the catheter 408 with the tissue piercing structure. With the closure device 300 releasably engaging the tissue piercing structure 554, the tissue piercing structure 554 may be advanced and rotated (for example, about ¼ of a turn) relative to the loading collar, to stretch and radially compress the closure device 300, as described above. The tissue piercing structure is advanced through the septum secundum 442 and septum primum 444, as shown in FIGS. 10-12. In some embodiments, tissue piercing structure 554 may be advanced across the septa manually. In other embodiments, tissue piercing structure 554 may be advanced across the septum using a spring loaded handle. In one embodiment, while penetrating the septa the tissue piercing structure and loading collar are also rotated to assist in penetration.

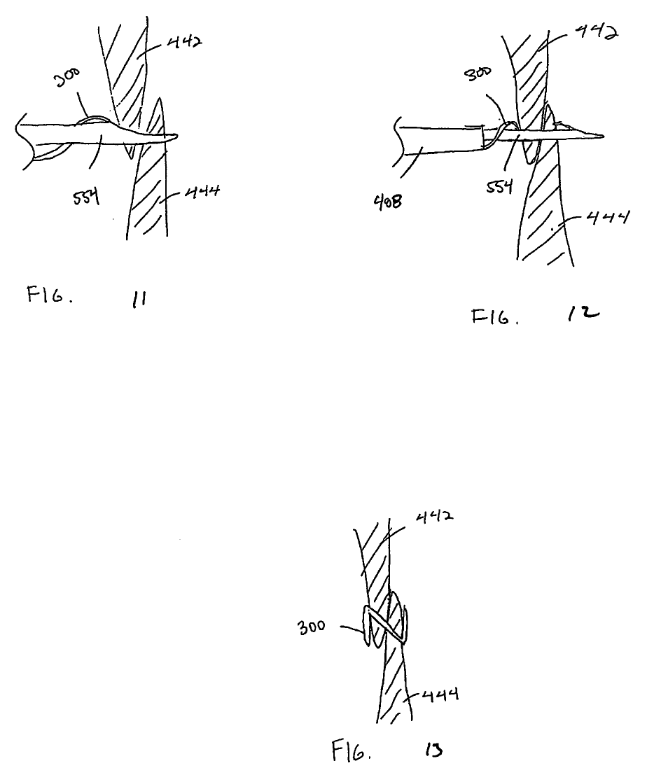

In one embodiment, the tissue piercing structure is advanced such that at least one helical section of the closure device crosses the septa. After optimal positioning is achieved, the closure device 300 can be released and the catheter 408 can be removed, as shown in FIG. 12. Release of the closure device 300 may occur, for example, by proximally retracting the tissue piercing structure 554, as described above. Rotation of the tissue piercing structure 554, relative to the loading collar 778, allows the implant 300 to relax to its natural diameter. Retraction of the tissue piercing structure 554 past the septum primum and septum secundum allows the distal tang 312 to exit the opening 558 of the tissue piercing structure. Finally, rotation of the loading collar relative to the implant allows the proximal end of the closure device 300 to snap out of the loading collar 778, releasing the device 300. When release is complete, the closure device should be in or biased towards its natural state, preferably radially expanded and axially contracted. This allows the closure device to pinch together the septum primum and septum secundum to close the patent foramen ovale. This thereby results in a continual closure force on both the septum primum and septum secundum.

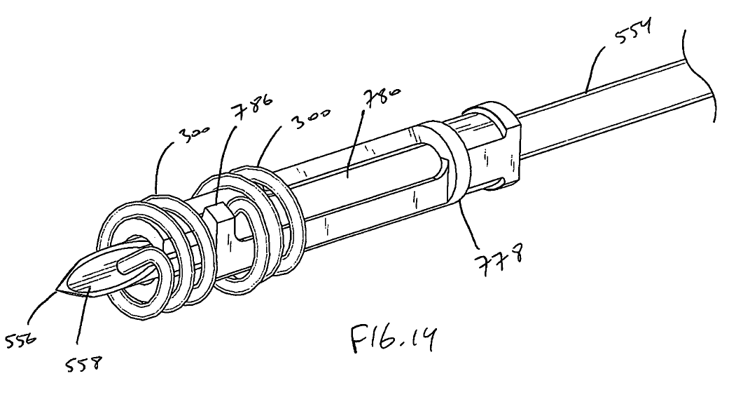

In one embodiment, multiple closure devices may be delivered during the same procedure by providing two or more closure devices on the loading collar. Any number of closure devices may be delivered in this manner. FIG. 14 shows loading collar 778 having a plurality of closure devices 300 provided thereon. The distal most device 300 is loaded onto the tissue piercing structure 554 and loading collar 778 as described with respect to FIGS. 6 and 7 above. Additional closure devices are provided on the loading collar 778, proximal of the projection 786, with proximal tang 314 and distal tang 312 of each device extending into the opening 780.

After delivery of the distal most device 300 as described above, the tissue piercing structure 554 can be retracted proximally into the loading collar 778 until the distal tang 312 of the next device 300 snaps into the opening 558 of the tissue piecing structure. The tissue piercing structure is then advanced, pulling the device 300 along the open slot 780 of the loading collar 778. By rotating and advancing the tissue piercing structure 554, the distal tang is guided through the track 788 until the device again sits at the distal end of the loading collar. The device is then deployed as previously described.

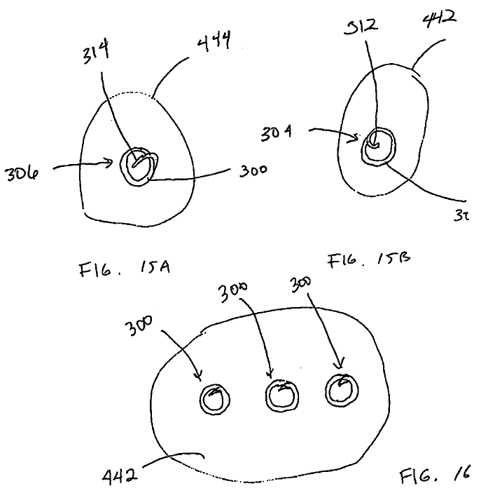

FIG. 15A shows the distal end 306 of the closure device 300 delivered at the septum primum 444 and FIG. 15B shows the proximal end 304 of the closure device 300 delivered at the septum secundum 442. Material exposure in both the right and, more importantly, the left atria is minimal, thereby reducing the risk of clot formation. FIG. 16 shows an embodiment wherein a plurality of closure devices 300 are delivered to the septal defect, such as by using the devices described above. As shown in FIG. 16, three closure devices 300 extend through the septa of the patent foramen ovale, each adjacent to one another. Although the closure devices of FIG. 16 are shown in a linear arrangement, it will be appreciated that other configurations may be used to adequately close the patent foramen ovale. In one embodiment, closure devices may be first deployed at the corners of the mouth of the patent foramen ovale, and then across the tunnel of the patent foramen ovale.

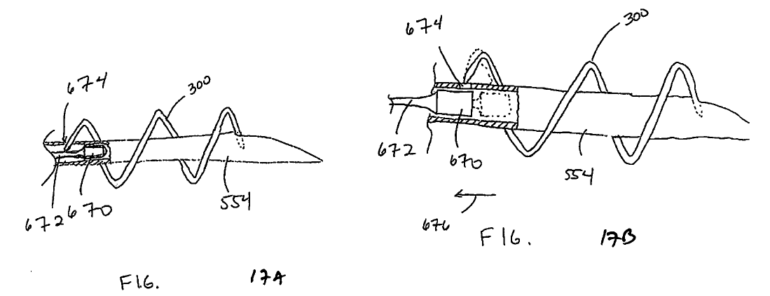

FIGS. 17A and 17B illustrate another embodiment of a delivery device used to deliver a closure device 300 such as described above. A tissue piercing structure 554 is provided similar to that described above, having an opening 558 for releasably engaging a distal tang 312 of closure device 300. Tissue piercing structure 554 also includes a loading portion, which may include an opening 674 proximal to the opening 558 for releasably engaging a proximal tang 314 of closure device 300. As illustrated, the loading portion is integral with the tissue piercing structure, although in another embodiment, it may be formed from a separate piece. An actuator 672 extends through the lumen of the tissue piercing structure, and preferably includes a release element 670 at a distal end thereof. When the actuator 672 and release element 670 are actuated in the direction of arrow 676, as shown in FIG. 17B, the proximal end of the closure device 300 is pressed out of the opening 674 and tissue piercing structure 554. In some embodiments, the actuator and release element may be actuated in the direction opposite the direction of arrow 676 to release the closure device 300 from the tissue piercing structure 554.

In some embodiments, the closure device 300 may be stretched out along the length of the tissue piercing structure 554 and may be rotated axially, thereby reducing the diameter of the closure device. The closure device may be delivered in a manner similar to that described above, wherein the tissue piercing element 558 penetrates the septa of a patent foramen ovale. Then, the release element 670 may be actuated to release the closure device 300, and the tissue piercing element can be proximally retracted. The closure device upon being released returns to its natural state, pinching the septa of the patent foramen ovale together.

In some embodiments, radiopaque markers may be provided on the tissue piercing structure 554 or delivery device 400 to aid in placement at the treatment site. In some embodiments, the radiopaque markers are crimped on to the tissue piercing structure or delivery device. In one embodiment, the radiopaque markers are tubular bands crimped on to the tissue piercing structure of delivery device. In some embodiments, the radiopaque markers are coatings applied to the structure or device. In some embodiments, the radiopaque markers may be platinum or iridium, and the like.

While particular forms of the invention have been described, it will be apparent that various modifications can be made without departing from the spirit and scope of the invention. Accordingly, it is not intended that the invention be limited, except as by the appended claims.

1. A method of closing a patent foramen ovale having a septum primum and a septum secundum, comprising:
delivering an elongate body having a proximal end and a distal end to the patent foramen ovale, the elongate body having a tissue piercing structure at its distal end and a coil releasably engaged with the elongate body;
advancing the tissue piercing structure and the coil through the septa of the patent foramen ovale; and
releasing the coil from the elongate body and withdrawing the tissue piercing structure from the septa of the patent foramen ovale, wherein the coil when released contracts to pinch the septum primum and the septum secundum together.

2. The method of claim 1, wherein the elongate body includes an opening near its distal end.

3. The method of claim 2, wherein the coil has a distal end that releasably engages the opening in the elongate body near its distal end.

4. The method of claim 3, wherein a loading portion releasably engages a proximal end of the coil, the coil being advanced through the patent foramen ovale while the coil is engaged with both the loading portion and the opening near the distal end of the elongate body to axially elongate and radially reduce the coil.

5. The method of claim 1, further comprising delivering a loading collar with the elongate body to the patent foramen ovale, the loading collar releasably engaging a proximal end of the coil.

6. The method of claim 5, wherein the elongate body is rotatable relative to the loading collar.

7. The method of claim 5, wherein the elongate body is axially slideable relative to the loading collar.

8. The method of claim 5, wherein the elongate body is advanced relative to the loading collar prior to advancing the coil to axially elongate the coil.

9. The method of claim 1, wherein the elongate body is delivered through an outer catheter.

10. The method of claim 1, wherein the tissue piercing structure and the coil are delivered first through the septum secundum and then through the septum primum.

11. The method of claim 1, wherein the coil is a first coil, and further comprising, after releasing the first coil from the elongate body and withdrawing the tissue piercing structure from the septa of the patent foramen ovale:
advancing the tissue piercing structure and a second coil releasably engaged with the elongate body through the septa of the patent foramen ovale at a location adjacent to the first coil; and
releasing the second coil from the elongate body and withdrawing the tissue piercing structure from the septa of the patent foramen ovale, wherein the second coil when released contracts to pinch the septum primum and the septum secundum together.

12. A method of closing a patent foramen ovale having a septum primum and septum secundum, comprising advancing a plurality of coils at least partially through the septa of the patent foramen ovale to secure the septum primum and septum secundum together.

13. The method of claim 12, wherein the plurality of coils are advanced sequentially through a single catheter.

14. The method of claim 12, wherein the plurality of coils are each advanced first through the septum secundum and then through the septum primum.

15. The method of claim 12, wherein the plurality of coils are each advanced first through the septum primum and then through the septum secundum.

16. The method of claim 12, wherein each of the coils is provided over a single elongate body and is advanced through the patent using a tissue piercing structure on the distal end of the elongate body.

17. The method of claim 12, wherein each of the coils after being advanced through the septa of the patent foramen ovale has a first end in the septum primum and a second end in the septum secundum.

18. The method of claim 12, wherein each of the coils after being advanced through the septa of the patent foramen ovale has a first end in the left atrium and a second end in the right atrium.

19. The method of claim 12, comprising advancing at least three coils through the septa of the patent forman ovale.

20. An assembly for delivering a coil through tissue in a patient, comprising:
a loading portion adapted to releasably engage a proximal end of the coil; and
a tissue piercing structure adapted to releasably engage a distal end of the coil, wherein the loading portion holds the coil relative to the tissue piercing structure to axially elongate and radially reduce the coil.

21. The assembly of claim 20, wherein the loading portion is integral with the tissue piecing structure.

22. The assembly of claim 21, wherein the loading portion comprises a slot adapted to receive the proximal end of the coil.

23. The assembly of claim 21, wherein the tissue piercing structure includes an opening adapted to releasably engage the distal end of the coil.

24. The assembly of claim 20, wherein the loading portion comprises a loading collar, and the tissue piecing structure is moveable relative to the loading collar to axially advance and rotate the distal end of the coil relative to the proximal end of the coil to axially elongate the coil.

25. The assembly of claim 24, wherein the tissue piercing structure is provided on an elongate body having a proximal end and a distal end, the elongate body extending through the loading collar.

26. The assembly of claim 20, further comprising a coil having a proximal end releasably engaging the loading portion and a distal end releasably engaging the tissue piercing structure.

27. The assembly of claim 26, wherein the proximal end of the coil comprises a tang that extends into a diameter defined by the coil.

28. The assembly of claim 26, wherein the distal end of the coil comprises a tang that extends into a diameter defined by the coil.

29. The assembly of claim 20, wherein the coil is sized to extend through a septum primum and a septum secundum of a patent foramen ovale.

30. The assembly of claim 20, wherein the loading portion is adapted to releasably engage a plurality of coils.
